(19) United States
(12) Patent Application Publication
(10) Pub. No.: US 2008/0229470 A1
(43) Pub. Date: Sep. 25, 2008

(54) Cap Having Auxiliary Visor

(76) Inventor: Sung-Gyu Kim, Busan (KR)

Correspondence Address: IPLA P.A., 3580 WILSHIRE BLVD., 17TH FLOOR, LOS ANGELES, CA, 90010, US

(21) Appl. No.: 12/064,205

(22) PCT Filed: May 15, 2006
(86) PCT No.: PCT/KR06/01814

§ 371 Date: Feb. 19, 2008

(30) Foreign Application Priority Data
Aug. 16, 2005 (KR) 10-2005-0074700

(51) Int. Cl. A61F 9/00; A42B 1/00

(52) U.S. Cl. 2/12; 2/209.3

(57) ABSTRACT

Disclosed is a cap having an auxiliary visor. The cap includes a hair band having enough elasticity to be worn on a user's head, a front visor coupled to the front of the hair band so as to block sunlight, and at least one auxiliary visor hinge-coupled to the top or bottom surface of the front visor and fixed thereto by an attachment/detachment means. The auxiliary visor is adapted to selectively unfold so as to shield the side of the user's face from UV rays, as well as fold so as to overlap the front visor for an aesthetic appearance.

### TECHNICAL FIELD

The present invention relates to a cap having an auxiliary visor, and more particularly to a cap having a front visor and an auxiliary visor hinge-coupled to the front visor so as to fold on the front visor or unfold from it when the side of a user's face is to be shielded.

### BACKGROUND ART

In general, caps are worn on user's heads during outdoor activities in order to block the sunlight, for example, during mountain climbing or fishing, or on a beach or playground. A cap includes a hair band having enough elasticity to be worn on the head and a front visor connected to both sides of the front of the hair band by a hinge unit in an angle-adjustable manner so as to block the sunlight.

However, conventional caps have a problem in that, although they can block sunlight shining on the front of user's face, they cannot block sunlight shining on the side of the face during outdoor activities (e.g. watching sports) and leisure activities.

A conventional cap disclosed in a pertinent patent publication will now be described briefly.

Korean Utility Model Registration No. 20-246437 discloses a cap, entitled V-BLOCKING CAP HAVING AUXILIARY VISOR.

The cap disclosed in the above publication (for clarity, not shown in the drawings) includes a hair band having enough elasticity to be worn on the head, a visor connected to both sides of the front of the hair band by a hinge unit in an angle-adjustable manner, and an auxiliary visor detachably connected to both sides of the back of the hair band by a snap button so as to shield the user's neck. The visor and the auxiliary visor are made of a transparent material, particularly tinted UV-blocking PET film.

The conventional cap still has a problem in that it can only shield the nape of the neck, but not the side of the user's face.

### DISCLOSURE OF INVENTION

### Technical Problem

The conventional caps have a problem in that, although they can block sunlight shining on the front of user's face, they cannot block sunlight shining on the side of the face during outdoor activities (e.g. watching sports) and leisure activities.

The conventional cap still has a problem in that it can only shield the nape of the neck, but not the side of the user's face.

### Technical Solution

Therefore, the present invention has been made in view of the above-mentioned problems, and it is an object of the present invention to provide a cap having an auxiliary visor capable of shielding the side of a user's face, as well as the front, so that UV rays are blocked as desired.

According to an aspect of the present invention, there is provided a cap having an auxiliary visor, the cap including a hair band to be worn on a user's head, a front visor coupled to a front portion of the hair band so as to block sunlight, and at least one auxiliary visor hinge-coupled to a top surface of the front visor and fixed to the top surface by an attachment/detachment means.

Preferably, the cap further includes at least one auxiliary visor hinge-coupled to a bottom surface of the front visor and fixed to the bottom surface by an attachment/detachment means.

According to another aspect of the present invention, there is provided a cap having an auxiliary visor, the cap including a hair band to be worn on a user's head, a front visor coupled to a front portion of the hair band so as to block sunlight, a plurality of upper auxiliary visors hinge-coupled to a top surface of the front visor and fixed to the top surface by an attachment/detachment means, and a plurality of lower auxiliary visors hinge-coupled to a bottom surface of the front visor and fixed to the bottom surface by an attachment/detachment means.

According to still another aspect of the present invention, there is provided a cap having an auxiliary visor, the cap including a hair band to be worn on a user's head, a front visor coupled to a front portion of the hair band so as to block sunlight, an upper auxiliary visor hinge-coupled to a side of a top surface of the front visor and fixed to the side by an attachment/detachment means, and a lower auxiliary visor hinge-coupled to a side of a bottom surface of the front visor and fixed to the side by an attachment/detachment means, the upper and lower auxiliary visors being positioned diagonally.

### ADVANTAGEOUS EFFECTS

According to the present invention provides, the cap having an auxiliary visor can selectively shield the side of a user's face from UV rays by unfolding the auxiliary visor, which can be folded on the front visor for an aesthetic appearance.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a perspective view showing a cap according to a first embodiment of the present invention;

FIG. 2 is a perspective view showing a cap according to a second embodiment of the present invention;

FIG. 3 is a perspective view showing a cap, from below, according to a third embodiment of the present invention;

FIG. 4 is a perspective view showing a cap, from below, according to a fourth embodiment of the present invention;

FIG. 5 is a perspective view showing a cap according to a fifth embodiment of the present invention;

FIG. 6 is a perspective view showing a cap according to a sixth embodiment of the present invention; and

FIG. 7 shows an example of use of the cap according to the first embodiment of the present invention.

### BEST MODE FOR CARRYING OUT THE INVENTION

Hereinafter, a preferred embodiment of the present invention will be described with reference to the accompanying drawings. In the following description and drawings, the same reference numerals are used to designate the same or similar components, and so repetition of the description on the same or similar components will be omitted.

Caps according to first to sixth embodiments of the present invention basically include a hair band 4 having enough elasticity to be worn on user's head and a front visor 2 connected to the front of the hair band 4 so as to block the sunlight, as in the case of conventional caps.

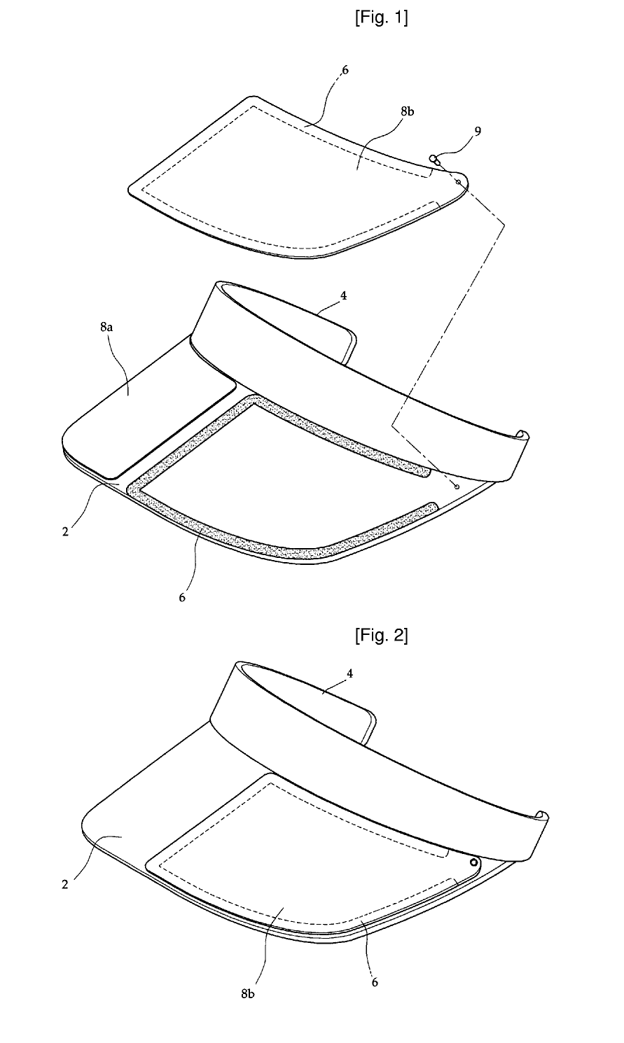

FIG. 1 is a perspective view showing a cap according to a first embodiment of the present invention.

Referring to the drawing, a plurality of auxiliary visors 8a and 8b are positioned on the top surface of the front visor 2. The auxiliary visors 8a and 8b are obtained by bisecting a visor, which has the same shape as the front visor 2, along its central line. A rear portion of each auxiliary visor 8a and 8b is hinge-coupled to the front visor 2 by a rivet.

Preferably, the plurality of auxiliary visors 8a and 8b exactly overlap the front visor 2 when folded.

When unfolded, as shown in FIG. 7, the auxiliary visors 8a and 8b are positioned along the rim of the front visor 2 so that the area shielded from the sunlight is increased by at least 1.5 times on both sides.

The front visor 2 and the auxiliary visors 8a and 8b have corresponding attachment/detachment means 6 positioned on the top and bottom surfaces thereof, respectively, so that the auxiliary visors 8a and 8b can be fastened to the top surface of the front visor 2.

As the attachment/detachment means 6, Velcro fasteners or snap fasteners are preferably used (Velcro fasteners are most preferred). The attachment/detachment means 6 may extend along a part of the rim of the auxiliary visors 8a and 8b or along the entire rim, if necessary.

Although a plurality of auxiliary visors 8a and 8b are positioned on both sides of the top surface of the front visor 2 in the present embodiment, it is also possible that only one auxiliary visor is positioned to the left or right.

### MODE FOR THE INVENTION

FIG. 2 is a perspective view showing a cap according to a second embodiment of the present invention.

Referring to the drawing, an auxiliary visor 8b is positioned on one side of the top surface of the front visor 2 so as to shield one of both sides of the user's face from the sunlight, based on his preference.

As in the case of the previous embodiment, Velcro fasteners are positioned on the top and bottom surfaces of the front and auxiliary visors 2 and 8b, respectively, as attachment/detachment means 6.

The auxiliary visor may be positioned on the bottom surface of the front visor 2.

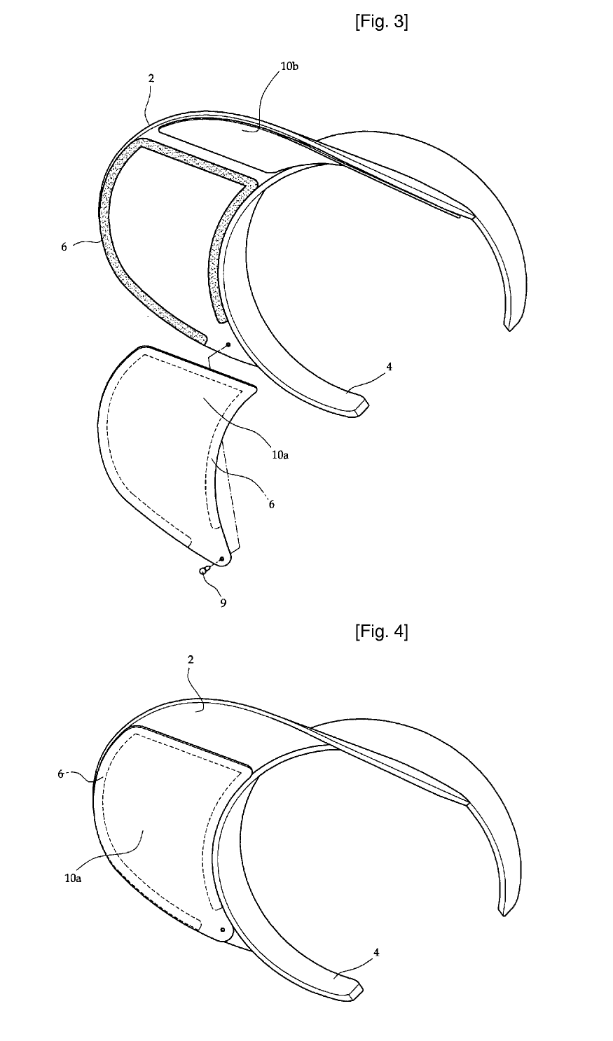

FIG. 3 is a perspective view showing a cap, from below, according to a third embodiment of the present invention.

Referring to the drawing, a plurality of auxiliary visors 10a and 10b are positioned on the bottom surface of the front visor 2. The auxiliary visors 10a and 10b are obtained by bisecting a visor, which has the same shape as the front visor 2, along its central line. A rear portion of each auxiliary visor 10a and 10b is hinge-coupled to the front visor 2 by a rivet 9.

The front visor 2 and the auxiliary visors 10a and 10b have corresponding attachment/detachment means 6 positioned on the bottom and top surfaces thereof, respectively, so that the auxiliary visors 10a and 10b can be fastened to the bottom surface of the front visor 2.

Velcro fasteners are preferably used as the attachment/detachment means 6, as mentioned above.

The auxiliary visors 10a and 10b, which are positioned on the bottom surface of the front visor 2, preferably overlap the front visor 2 when folded on the bottom surface so that they are hidden from the outside for an aesthetic appearance.

Although a plurality of auxiliary visors 10a and 10b are positioned on both sides of the bottom surface of the front visor 2 in the present embodiment, it is also possible that only one auxiliary visor is positioned to the left or right.

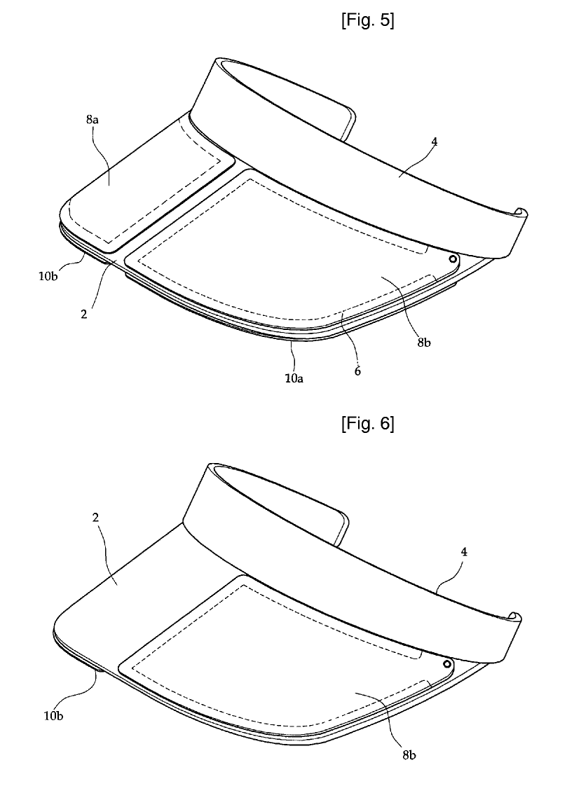

FIG. 4 is a perspective view showing a cap according to a fourth embodiment of the present invention.

Referring to the drawing, an auxiliary visor 10b is positioned on one side of the bottom surface of the front visor 2 so as to shield one of both sides of the user's face from the sunlight, based on his preference.

As mentioned above, Velcro fasteners are positioned on the bottom and top surfaces of the front and auxiliary visors 2 and 10a, respectively, as attachment/detachment means 6.

FIG. 5 is a perspective view showing a cap according to a fifth embodiment of the present invention.

Referring to the drawing, a plurality of upper and lower auxiliary visors 8a, 8b, 10a, and 10b are positioned on the top and bottom surfaces of the front visor 2, respectively. The auxiliary visors are obtained by bisecting a visor, which has the same shape as the front visor 2, along its central line and are hinge-coupled to the front visor 2.

When folded on the top and bottom surfaces of the front visor 2, respectively, the upper and lower auxiliary visors 8a, 8b, 10a, and 10b overlap the front visor 2. When unfolded, they are positioned side by side so that the area shielded by them doubles.

One or both of the upper and lower auxiliary visors 8a, 8b, 10a, and 10b may be unfolded from the top and bottom surfaces of the front visor 2, respectively, as desired.

The front visor 2 and the upper and lower auxiliary visors 8a, 8b, 10a, and 10b have corresponding attachment/detachment means 6 positioned on both top and bottom surfaces so that the upper and lower auxiliary visors 8a, 8b, 10a, and 10b can be fastened to the top and bottom surfaces of the front visor 2, respectively.

As the attachment/detachment means 6, Velcro fasteners or snap fasteners are preferably used (Velcro fasteners are most preferred).

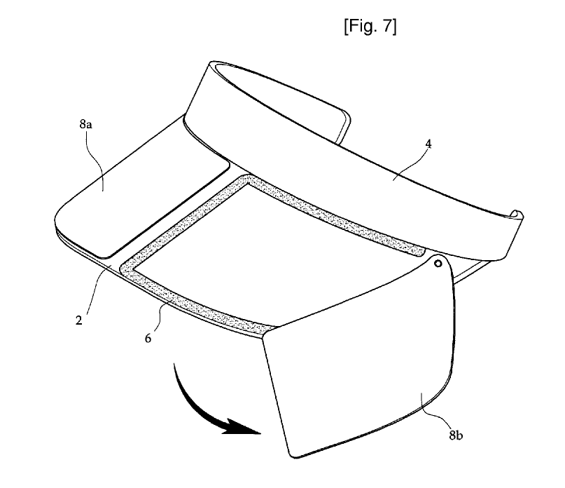

FIG. 6 is a perspective view showing a cap according to a sixth embodiment of the present invention.

Referring to the drawing, upper and lower auxiliary visors 8b and 10b are hinge-coupled to one side of the top surface of the front visor 2 and to one side of the bottom surface thereof, respectively, in a diagonal configuration.

For example, the upper auxiliary visor 8b is positioned to the left on the top surface of the front visor 2, and the lower auxiliary visor 10b is positioned to the right on the bottom surface thereof.

In this case, the upper and lower auxiliary visors 8b and 10b are unfolded so as to shield the left and right of the face, respectively.

The upper and lower auxiliary visors 8b and 10b have a size and a shape corresponding to a half of the front visor 2, as mentioned above, so that they are folded on the top and bottom surfaces of the front visors 2, respectively, for an aesthetic appearance.

The upper and lower auxiliary visors 8b and 10b and the front visor 2 have attachment/detachment means 6, as mentioned above, so as to remain in a fixed condition.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

For example, the present invention is applicable to all types of caps.

### INDUSTRIAL APPLICABILITY

As can be seen from the foregoing, according to the present invention provides, the cap having an auxiliary visor can selectively shield the side of a user's face from UV rays by unfolding the auxiliary visor, which can be folded on the front visor for an aesthetic appearance.

1. A cap having an auxiliary visor comprising:
a hair band to be worn on a user's head; a front visor coupled to a front portion of the hair band so as to block sunlight; and
at least one auxiliary visor hinge-coupled to a top surface of the front visor and fixed to the top surface by an attachment/detachment means.

2. A cap having an auxiliary visor comprising:
a hair band to be worn on a user's head; a front visor coupled to a front portion of the hair band so as to block sunlight; and
at least one auxiliary visor hinge-coupled to a bottom surface of the front visor and fixed to the bottom surface by an attachment/detachment means.

3. A cap having an auxiliary visor comprising:
a hair band to be worn on a user's head;
a front visor coupled to a front portion of the hair band so as to block sunlight;
a plurality of upper auxiliary visors hinge-coupled to a top surface of the front visor and fixed to the top surface by an attachment/detachment means: and
a plurality of lower auxiliary visors hinge-coupled to a bottom surface of the front visor and fixed to the bottom surface by an attachment/detachment means.

4. A cap having an auxiliary visor comprising:
a hair band to be worn on a user's head; a front visor coupled to a front portion of the hair band so as to block sunlight:
an upper auxiliary visor hinge-coupled to a side of a top surface of the front visor and fixed to the side by an attachment/detachment means; and
a lower auxiliary visor hinge-coupled to a side of a bottom surface of the front visor and fixed to the side by an attachment/detachment means, the upper and looser auxiliary visors being positioned diagonally.

5. The cap having an auxiliary visor as claimed in claim 1, wherein the auxiliary visor has a shape corresponding to a half of the front visor and is hinge-coupled to the front visor by a rivet.

6. The cap having an auxiliary visor as claimed in claim 1, wherein the attachment/detachment means is a Velcro fastener or snap fastener.

7. The cap having an auxiliary visor as claimed in claim 2, wherein the auxiliary visor has a shape corresponding to a half of the front visor and is hinge-coupled to the front visor by a rivet.

8. The cap having an auxiliary visor as claimed in claim 2, wherein the attachment/detachment means is a Velcro fastener or snap fastener.

9. The cap having an auxiliary visor as claimed in claim 3, wherein the attachment/detachment means is a Velcro fastener or snap fastener.

10. The cap having an auxiliary visor as claimed in claim 4, wherein the attachment/detachment means is a Velcro fastener or snap fastener.
